(19) United States
(12) Patent Application Publication
(10) Pub. No.: US 2009/0076386 A1
(43) Pub. Date: Mar. 19, 2009

(54) METHOD AND SYSTEM FOR ACQUIRING VOLUME OF INTEREST BASED ON POSITIONAL INFORMATION

(75) Inventors: Sharathchander Sirivolu, Waukesha, WI (US); Michael Joseph Washburn, Brookfield, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/855,668

(22) Filed: Sep. 14, 2007

(51) Int. Cl. A61B 8/00

(52) U.S. Cl. 600/437

(57) ABSTRACT

A method for imaging based on a position of an image acquisition device is presented. The method includes obtaining a first desired image data set representative of a first desired image, where the first desired image data set is acquired at a first position of the image acquisition device. Further, the method includes recording positional information corresponding to the first position of the image acquisition device. In addition, the method includes obtaining a second desired image data set representative of a second desired image, where the second desired image data set is acquired at a second position of an image acquisition device. The method also includes recording positional information corresponding to the second position of the image acquisition device. Moreover, the method includes acquiring image data between the first position and the second position of the image acquisition device. Systems and computer-readable medium that afford functionality of the type defined by this method is also contemplated in conjunction with the present technique.

### BACKGROUND

The invention relates generally to methods and apparatus for review of medical imaging exams, and more particularly to methods and apparatus for review of image data, such as that resulting from ultrasound exams.

Ultrasound imaging (also referred to as ultrasound scanning or sonography) is a relatively inexpensive and radiation-free imaging modality. As will be appreciated, ultrasound typically involves non-invasive imaging and is being increasingly used in the diagnosis of a number of organs and conditions, without X-ray radiation. Further, modern obstetric medicine for guiding pregnancy and childbirth is known to rely heavily on ultrasound to provide detailed images of the fetus and the uterus. In addition, ultrasound is also extensively used for evaluating the kidneys, liver, pancreas, heart, and blood vessels of the neck and abdomen. More recently, ultrasound imaging and ultrasound angiography are finding a greater role in the detection, diagnosis and treatment of heart disease, heart attack, acute stroke and vascular disease which may lead to stroke. Also, ultrasound is also being used more and more to image the breasts and to guide biopsy of breast cancer.

However, a drawback of the currently available techniques is that these procedures are extremely tedious and time-consuming. Also, use of these techniques calls for a high level of skill and experience of a clinician to acquire images of good quality and enable accurate diagnoses. Furthermore, use of the currently available ultrasound imaging systems entails selection of the volume angle by a user, such as the clinician. The user-selected volume angle may then be used to determine a sweep angle of an image acquisition device such as a probe. This computation of the sweep angle of the probe may disadvantageously lead to the acquisition of an undesirable image volume. More particularly, the sweep angle so determined may lead to the acquisition of an image volume that is relatively larger than a desired image volume. Alternatively, an image volume that is substantially smaller than the desired image volume may be acquired. Furthermore, acquisition of undesirable image data may call for a repeat scan with a different volume angle.

There is therefore a need for a system for the acquisition of a desirable image data set representative of anatomical regions of interest. In particular, there is a significant need for a design that advantageously facilitates the acquisition of a desired image volume thereby substantially reducing need repeat scans and enhancing the clinical workflow.

### BRIEF DESCRIPTION

In accordance with aspects of the present technique, a method for imaging based on a position of an image acquisition device is presented. The method includes obtaining a first desired image data set representative of a first desired image, where the first desired image data set is acquired at a first position of the image acquisition device. Further, the method includes recording positional information corresponding to the first position of the image acquisition device. In addition, the method includes obtaining a second desired image data set representative of a second desired image, where the second desired image data set is acquired at a second position of the image acquisition device. The method also includes recording positional information corresponding to the second position of the image acquisition device. Moreover, the method includes acquiring image data between the first position and the second position of the image acquisition device. Computer-readable medium that afford functionality of the type defined by this method is also contemplated in conjunction with the present technique.

In accordance with yet another aspect of the present technique, a method for imaging based on a position of an image acquisition device is presented. The method includes selecting acquisition parameters. Furthermore, the method includes obtaining a first desired image and a second desired image based on the selected acquisition parameters. Additionally, the method includes recording a first position and a second position of the image acquisition device, where the first position of the image acquisition device is associated with the first desired image and the second position of the image acquisition device is associated with the second desired image. The method also includes acquiring image data between the first position and the second position of the image acquisition device.

In accordance with further aspects of the present technique, a position sensing system is presented. The system includes a position sensing platform configured to facilitate acquisition of image data based on a first position and a second position of an image acquisition device, where the position sensing platform is configured to obtain a first desired image data set representative of a first desired image, where the first desired image data set is acquired at the first position of the image acquisition device, record positional information corresponding to the first position of the image acquisition device, obtain a second desired image data set representative of a second desired image, where the second desired image data set is acquired at a second position of the image acquisition device, record positional information corresponding to the second position of the image acquisition device, and acquire image data between the first position and the second position of the image acquisition device.

In accordance with further aspects of the present technique, a system for acquiring image data based on a position of an image acquisition device is presented. The system includes an image acquisition device configured to acquire image data representative of an anatomical region of interest. Additionally, the system includes a position sensing device in operative association with the image acquisition device and configured to provide positional information associated with the image acquisition device. Further, the system includes an imaging system in operative association with the image acquisition device and including an acquisition subsystem configured to acquire image data, where the image data is representative of the anatomical region of interest, and a processing subsystem in operative association with the acquisition subsystem and comprising a position sensing platform configured to facilitate moving the image acquisition device to at least a first desirable position and a second desirable position based on the acquired image data and positions of the image acquisition device.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary diagnostic system, in accordance with aspects of the present technique;

FIG. 2 is a block diagram of an exemplary imaging system in the form of an ultrasound imaging system for use in the exemplary diagnostic system of FIG. 1;

FIG. 3 illustrates a portion of a probe for use in the system illustrated in FIG. 1, in accordance with aspects of the present technique;

FIG. 4 is a block diagram of an exemplary position sensing system, in accordance with aspects of the present technique;

FIG. 5 is a front view of a user interface area of the exemplary diagnostic system of FIG. 1, in accordance with aspects of the present technique;

FIG. 6 is a front view of a display area of the exemplary diagnostic system of FIG. 1, in accordance with aspects of the present technique;

FIGS. 7A-7B are flow charts illustrating an exemplary process of acquiring a volume of interest based on positional information, in accordance with aspects of the present technique;

FIGS. 8-9 are diagrammatic illustrations of an exemplary process of acquiring a volume of interest based on positional information, in accordance with aspects of the present technique;

FIGS. 10A-10B are flow charts illustrating another exemplary process of acquiring a volume of interest based on positional information, in accordance with aspects of the present technique; and

FIGS. 11A-11B are flow charts illustrating yet another exemplary process of acquiring a volume of interest based on positional information, in accordance with aspects of the present technique.

### DETAILED DESCRIPTION

As will be described in detail hereinafter, a method of imaging based on positional information associated with an image acquisition device and a system for imaging based on positional information configured to optimize acquisition of a desirable volume of interest, simplify procedural workflow for imaging an anatomical region of interest in a patient and enhance the speed of procedural time taken to image the anatomical region of interest in the patient, are presented. Employing the method and system described hereinafter, patient comfort may be dramatically enhanced as the method of imaging entails acquisition of only a desirable volume of interest, thereby substantially reducing patient breath hold time.

Although the exemplary embodiments illustrated hereinafter are described in the context of a medical imaging system, it will be appreciated that use of the diagnostic system in industrial applications are also contemplated in conjunction with the present technique.

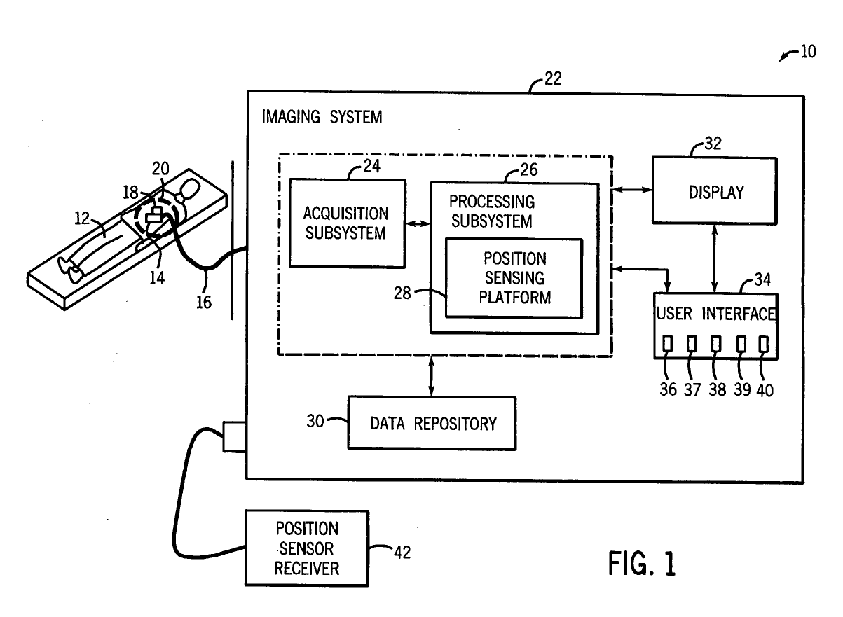

FIG. 1 is a block diagram of an exemplary system 10 for use in diagnostic imaging in accordance with aspects of the present technique. The system 10 may be configured to acquire image data from a patient 12 via an image acquisition device 14. In one embodiment, the image acquisition device 14 may include a probe, where the probe may include an invasive probe, or a non-invasive or external probe, such as an external ultrasound probe, that is configured to aid in the acquisition of image data. By way of example, the image acquisition device 14 may include a probe, where the probe comprises an imaging catheter, an endoscope, a laparoscope, a surgical probe, an external probe, or a probe adapted for interventional procedures. More particularly, the image acquisition device 14 may include a probe configured to facilitate acquisition of an image volume. It may be noted that the terms probe and image acquisition device may be used interchangeably.

Reference numeral 16 may be representative of a probe cable configured to aid in operatively coupling the image acquisition device 14 to an imaging system. Although the present example illustrates the image acquisition device 14 as being coupled to an imaging system via the probe cable 16, it will be understood that the probe may be coupled with the imaging system via other means, such as wireless means, for example. Also, in certain other embodiments, image data may be acquired via one or more sensors (not shown) that may be disposed on the patient 12. By way of example, the sensors may include physiological sensors (not shown), such as electrocardiogram (ECG) sensors and/or positional sensors, such as electromagnetic field sensors or inertial sensors. These sensors may be operationally coupled to a data acquisition device, such as an imaging system, via leads (not shown), for example.

Additionally, the system 10 may include a position sensing device 18, where the position sensing device 18 may be configured to facilitate gathering of positional information associated with the image acquisition device 14. As used herein, the term positional information is used to represent positional coordinates of the image acquisition device 14 with reference to an anatomical region of interest under examination. In one embodiment, the position sensing device 18 may include a position sensor. Furthermore, the position sensing device 18 may be in operative association with the image acquisition device 14. Also, in one embodiment, the position sensing device 18 may be disposed adjacent to the image acquisition device 14, as depicted in FIG. 1. Reference numeral 20 is representative of a portion of image acquisition device 14, the position sensing device 18 and the probe cable 16.

The system 10 may also include a medical imaging system 22 that is in operative association with the image acquisition device 14. It should be noted that although the exemplary embodiments illustrated hereinafter are described in the context of a medical imaging system, other imaging systems and applications, such as industrial imaging systems and non-destructive evaluation and inspection systems, such as pipeline inspection systems and liquid reactor inspection systems, are also contemplated. Additionally, the exemplary embodiments illustrated and described hereinafter may find application in multi-modality imaging systems that employ ultrasound imaging in conjunction with other imaging modalities, position-tracking systems or other sensor systems. It may be noted that the other imaging modalities may include medical imaging systems, such as, but not limited to, an ultrasound imaging system, a computed tomography (CT) imaging system, a magnetic resonance (MR) imaging system, a nuclear imaging system, a positron emission topography system or an X-ray imaging system.

In a presently contemplated configuration, the medical imaging system 22 may include an acquisition subsystem 24 and a processing subsystem 26. Further, the acquisition subsystem 24 of the medical imaging system 22 may be configured to acquire image data representative of one or more anatomical regions of interest in the patient 12 via the image acquisition device 14. The image data acquired from the patient 12 may then be processed by the processing subsystem 26.

Additionally, the image data acquired and/or processed by the medical imaging system 22 may be employed to aid a clinician in identifying disease states, assessing need for treatment, determining suitable treatment options, and/or monitoring the effect of treatment on the disease states. It may be noted that the terms treatment and therapy may be used interchangeably. In certain embodiments, the processing subsystem 26 may be further coupled to a storage system, such as a data repository 30, where the data repository 30 may be configured to receive image data.

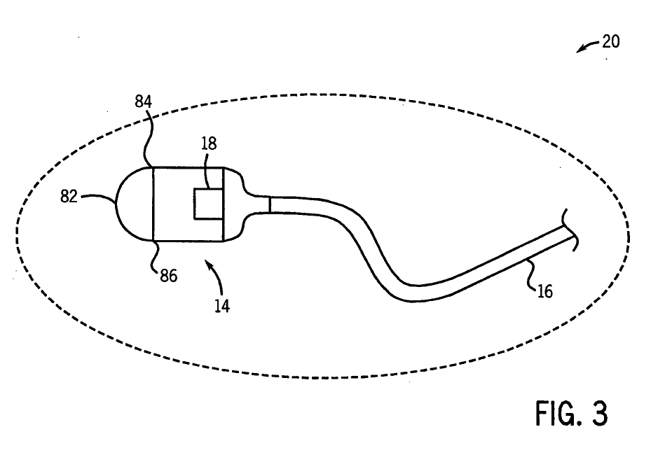

In accordance with exemplary aspects of the present technique, the processing subsystem 26 may include a position sensing platform 28 that is configured to aid in the acquisition of image data representative of anatomical regions of interest based on positional information associated with the image acquisition device 14. More particularly, the position sensing platform 28 may be configured to facilitate steering the image acquisition device 14 to at least a first desired location and a second desired location based on acquired image data and positions of the image acquisition device 14 and will be described in greater detail with reference to FIGS. 3-11.

Further, as illustrated in FIG. 1, the medical imaging system 22 may include a display 32 and a user interface 34. However, in certain embodiments, such as in a touch screen, the display 32 and the user interface 34 may overlap. Also, in some embodiments, the display 32 and the user interface 34 may include a common area. In accordance with aspects of the present technique, the display 32 of the medical imaging system 22 may be configured to display one or more images generated by the medical imaging system 22 based on the image data acquired via the image acquisition device 14, and will be described in greater detail with reference to FIGS. 3-11.

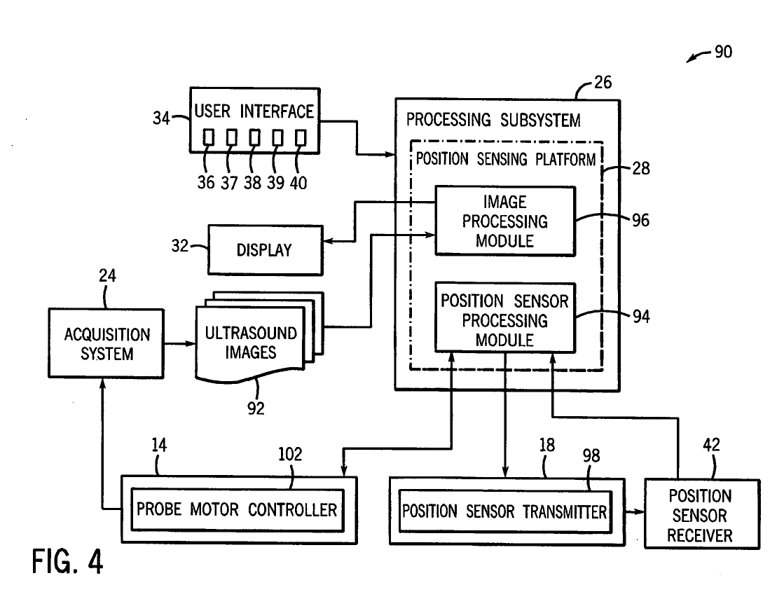

In addition, the user interface 34 of the medical imaging system 22 may include a human interface device (not shown) configured to facilitate the clinician in the acquisition of image data based on positional information associated with the image acquisition device 14. The human interface device may include a mouse-type device, a trackball, a joystick, a stylus, or buttons configured to aid the clinician in identifying the one or more regions of interest. However, as will be appreciated, other human interface devices, such as, but not limited to, a touch screen, may also be employed. Furthermore, in accordance with aspects of the present technique, the user interface 34 may be configured to aid the clinician in navigating through the images acquired by the medical imaging system 22. Additionally, the user interface 34 may also be configured to aid in manipulating and/or organizing the acquired image data for display on the display 32 and will be described in greater detail with reference to FIGS. 4-11.

Moreover, the position sensing device 18 may include a position sensor transmitter (not shown in FIG. 1), where the position sensor transmitter may be configured to communicate positional information associated with the position sensing device 18. More particularly, the position sensor transmitter may be configured to communicate the positional information associated with the position sensing device 18 to a position sensor receiver 42. In one embodiment, the position sensor transmitter may be disposed on the position sensing device 18. However, as will be appreciated, the position sensor transmitter may be disposed at other locations.

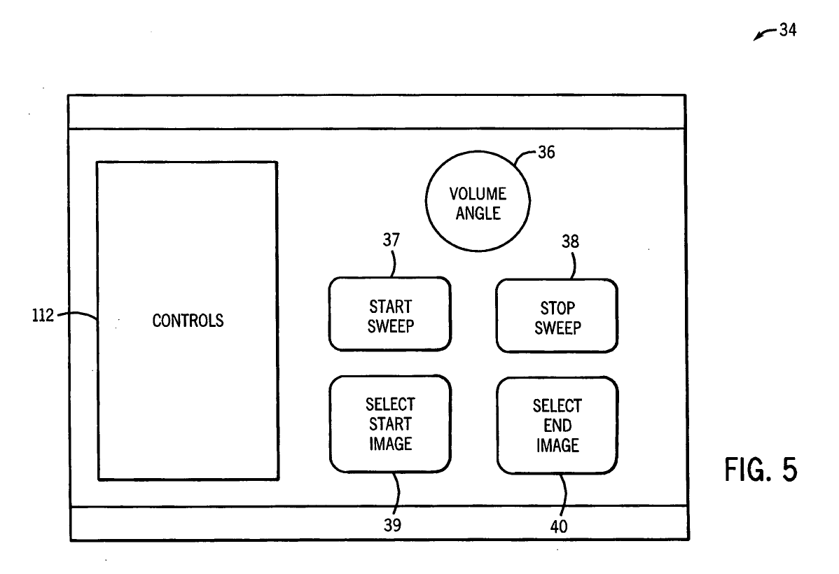

With continuing reference to FIG. 1, in accordance with exemplary aspects of the present technique, the user interface 34 may include a volume angle button 36, a start sweep button 37, a stop sweep button 38, a select start image button 39, and a select end image button 40. These buttons 36-40 may be configured to aid the clinician in the acquisition of image data based on the positional information of the image acquisition device 14. The working of the buttons 36-40 will be described in greater detail with reference to FIGS. 5-11.

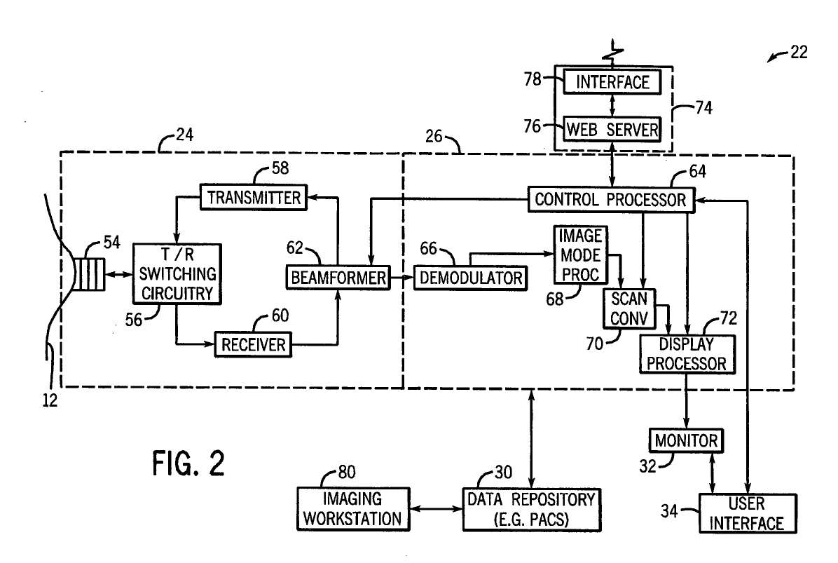

As previously noted, the medical imaging system 22 may include an ultrasound imaging system. FIG. 2 is a b lock diagram of an embodiment of the medical imaging system 22 of FIG. 1, where the medical imaging system 22 is shown as including an ultrasound imaging system 22. Furthermore, the ultrasound system 22 is shown as including the acquisition subsystem 24 and the processing subsystem 26, as previously described. The acquisition subsystem 24 may include a transducer assembly 54. In addition, the acquisition subsystem 24 includes transmit/receive (T/R) switching circuitry 56, a transmitter 58, a receiver 60, and a beamformer 62. In one embodiment, the transducer assembly 54 may be disposed in the image acquisition device 14 (see FIG. 1). Also, in certain embodiments, the transducer assembly 54 may include a plurality of transducer elements (not shown) arranged in a spaced relationship to form a transducer array, such as a one-dimensional or two-dimensional transducer array, for example. Additionally, the transducer assembly 54 may include an interconnect structure (not shown) configured to facilitate operatively coupling the transducer array to an external device (not shown), such as, but not limited to, a cable assembly or associated electronics. The interconnect structure may be configured to couple the transducer array to the T/R switching circuitry 56.

The processing subsystem 26 includes a control processor 64, a demodulator 66, an imaging mode processor 68, a scan converter 70 and a display processor 72. The display processor 72 is further coupled to a display monitor, such as the display 32 (see FIG. 1), for displaying images. User interface, such as the user interface 34 (see FIG. 1), interacts with the control processor 64 and the display 32. The control processor 64 may also be coupled to a remote connectivity subsystem 74 including a web server 76 and a remote connectivity interface 78. The processing subsystem 26 may be further coupled to the data repository 30 (see FIG. 1) configured to receive ultrasound image data, as previously noted with reference to FIG. 1. The data repository 30 interacts with an imaging workstation 80.

The aforementioned components may be dedicated hardware elements such as circuit boards with digital signal processors or may be software running on a general-purpose computer or processor such as a commercial, off-the-shelf personal computer (PC). The various components may be combined or separated according to various embodiments of the present technique. Thus, those skilled in the art will appreciate that the present ultrasound imaging system 22 is provided by way of example, and the present techniques are in no way limited by the specific system configuration.

In the acquisition subsystem 24, the transducer assembly 54 is in contact with the patient 12 (see FIG. 1). The transducer assembly 54 is coupled to the transmit/receive (T/R) switching circuitry 56. Also, the T/R switching circuitry 56 is in operative association with an output of the transmitter 58 and an input of the receiver 60. The output of the receiver 60 is an input to the beamformer 62. In addition, the beamformer 62 is further coupled to an input of the transmitter 58 and to an input of the demodulator 66. The beamformer 62 is also operatively coupled to the control processor 64 as shown in FIG. 2.

In the processing subsystem 26, the output of demodulator 66 is in operative association with an input of the imaging mode processor 68. Additionally, the control processor 64 interfaces with the imaging mode processor 68, the scan converter 70 and the display processor 72. An output of the imaging mode processor 68 is coupled to an input of the scan converter 70. Also, an output of the s can converter 70 is operatively coupled to an input of the display processor 72. The output of the display processor 72 is coupled to the display 32.

The ultrasound system 22 transmits ultrasound energy into the patient 12 and receives and processes backscattered ultrasound signals from the patient 12 to create and display an image. To generate a transmitted beam of ultrasound energy, the control processor 64 sends command data to the beamformer 62 to generate transmit parameters to create a beam of a desired shape originating from a certain point at the surface of the transducer assembly 54 at a desired steering angle. The transmit parameters are sent from the beamformer 62 to the transmitter 58. The transmitter 58 uses the transmit parameters to properly encode transmit signals to be sent to the transducer assembly 54 through the T/R switching circuitry 56. The transmit signals are set at certain levels and phases with respect to each other and are provided to individual transducer elements of the transducer assembly 54. The transmit signals excite the transducer elements to emit ultrasound waves with the same phase and level relationships. As a result, a transmitted beam of ultrasound energy is formed in the patient 12 along a scan line when the transducer assembly 54 is acoustically coupled to the patient 12 by using, for example, ultrasound gel. The process is known as electronic scanning.

In one embodiment, the transducer assembly 54 may be a two-way transducer. When ultrasound waves are transmitted into a patient 12, the ultrasound waves are backscattered off the tissue and blood samples within the patient 12. The transducer assembly 54 receives the backscattered waves at different times, depending on the distance into the tissue they return from and the angle with respect to the surface of the transducer assembly 54 at which they return. The transducer elements convert the ultrasound energy from the backscattered waves into electrical signals.

The electrical signals are then routed through the T/R switching circuitry 56 to the receiver 60. The receiver 60 amplifies and digitizes the received signals and provides other functions such as gain compensation. The digitized received signals corresponding to the backscattered waves received by each transducer element at various times preserve the amplitude and phase information of the backscattered waves.

The digitized signals are sent to the beamformer 62. The control processor 64 sends command data to beamformer 62. The beamformer 62 uses the command data to form a receive beam originating from a point on the surface of the transducer assembly 54 at a steering angle typically corresponding to the point and steering angle of the previous ultrasound beam transmitted along a scan line. The beamformer 62 operates on the appropriate received signals by performing time delaying and focusing, according to the instructions of the command data from the control processor 64, to create received beam signals corresponding to sample volumes along a scan line within the patient 12. The phase, amplitude, and timing information of the received signals from the various transducer elements are used to create the received beam signals.

The received beam signals are sent to the processing subsystem 26. The demodulator 66 demodulates the received beam signals to create pairs of I and Q demodulated data values corresponding to sample volumes along the scan line. Demodulation is accomplished by comparing the phase and amplitude of the received beam signals to a reference frequency. The I and Q demodulated data values preserve the phase and amplitude information of the received signals.

The demodulated data is transferred to the imaging mode processor 68. The imaging mode processor 68 uses parameter estimation techniques to generate imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 70. The scan converter 70 processes the parameter data by performing a translation from scan sequence format to display format. The translation includes performing interpolation operations on the parameter data to create display pixel data in the display format.

The scan converted pixel data is sent to the display processor 72 to perform any final spatial or temporal filtering of the scan converted pixel data, to apply grayscale or color to the scan converted pixel data, and to convert the digital pixel data to analog data for display on the display 32. The user interface 34 is coupled to the control processor 64 to allow a user to interface with the ultrasound system 22 based on the data displayed on the display 32.

FIG. 3 illustrates an enlarged view of the portion 20 (see FIG. 1) of the image acquisition device 14 (see FIG. 1). In one embodiment, the position sensing device 18 (see FIG. 1) may be disposed substantially close to a distal end of the image acquisition device 14, as depicted in FIG. 3. Furthermore, in the illustrated example, the image acquisition device 14 is shown as including a curvilinear probe face. It may be noted that use of probes with other types of probe faces are also contemplated in conjunction with the present technique. Reference numeral 82 may be representative of a central portion of the curvilinear probe face. Also, reference numeral 84 may be indicative of a first end of the curvilinear probe face, while a second end of the curvilinear probe face may be represented by reference numeral 86.

Turning no w to FIG. 4, a block diagram 90 of one embodiment of the diagnostic system 10 of FIG. 1 is depicted. As previously noted with reference to FIG. 1, the diagnostic system 10 may be configured to facilitate acquisition of image data based on positional information associated with the image acquisition device 14 (see FIG. 1). Accordingly, the system 10 may include the position sensing device 18 (see FIG. 1), where the position sensing device 18 is configured to aid in providing positional coordinates corresponding to the current locations of the image acquisition device 14. In a present example, the position sensing device 18 may include a position sensor, as previously noted. Also, in one embodiment, the position sensing device 18 may be operatively coupled to the image acquisition device 14. Further, the position sensing device 18 may include a position sensor transmitter 98 configured to facilitate transmission of the positional information of the image acquisition device 14 to a position sensor receiver, such as the position sensor receiver 42 (see FIG. 1), for example. Although, in the present example, the position sensing device 18 is shown as including the position sensor transmitter 98, it may be noted that the position sensor transmitter 98 may be separate from the position sensing device 18.

Furthermore, in a presently contemplated embodiment, the position sensing platform 28 (see FIG. 1) may include a position sensor processing module 94 and an image processing module 96. As illustrated in the example of FIG. 4, the position sensor receiver 42 is operatively coupled with the position sensor processing module 94. In one embodiment, the position sensor receiver 42 may be configured to communicate positional information associated with the image acquisition device 14 to the position sensor processing module 94. The position sensor processing module 94 may in turn be configured to utilize this positional information to aid in the acquisition of image data based on the positional information associated with the image acquisition device 14. More particularly, the position sensor processing module 94 may be configured to communicate the positional information to a probe motor controller 102 in the image acquisition device 14, which in turn may be configured to aid the clinician in steering the image acquisition device 14 to a desirable location to facilitate acquisition of image data representative of the anatomical region of interest. In other words, the clinician may steer the image acquisition device 14 to a desirable location, and image data, such as ultrasound image data 92, may be acquired by the acquisition subsystem 24, for example.

As noted hereinabove, the position sensing platform 28 may also include the image processing module 96. In one embodiment, the image processing module 96 may be configured to process the acquired image data, such a s the ultrasound image data 92, based on the positional information associated with the image acquisition device 14. For example, the image processing module 96 may be configured to obtain information regarding the position of the image acquisition device 14 from the position sensor processing module 94, and accordingly process the acquired image data 92 based on the obtained positional information. Additionally, the image processing module 96 may be configured to facilitate visualization of the acquired image data on the display 32, for example.

In addition, the diagnostic system 10 may also include the user interface 34. The user interface 34 may be operatively coupled with the processing subsystem 26, where the user interface 34 may be configured to facilitate acquisition of image data based on the positional information associated with the image acquisition device 14. More particularly, using the user interface 34, information associated with a start of the volume sweep, an end of the volume sweep, and the volume angle may be communicated to the position sensing platform 28 to aid in the acquisition of image data between the start and end of the volume sweep. The working of the position sensing system 90 will be described in greater detail with reference to FIGS. 5-11.

It may be noted t hat u se of the presently available techniques typically entails the clinician selecting a volume angle, which is then used to determine the sweep angle of the probe 14. Unfortunately, this determination of the sweep angle of the probe 14 may lead to acquisition of an undesirable volume, thereby resulting in the clinician working with an image volume that is larger than necessary or an image volume that is smaller than a desired image volume. Consequently, a different volume angle may have to be selected and the scan may have to be repeated, thereby adversely affecting the clinical workflow and causing patient discomfort.

In accordance with exemplary aspects of the present technique, the diagnostic system 10 (see FIG. 1) may be configured to allow the clinician to preview a first image and a second image in a volume sweep, thereby circumventing the disadvantages associated with the currently available techniques. In other words, by previewing the first and second images in the volume sweep, uncertainty associated with an acquired image volume may be removed. It may be noted that the first image may be representative of a start image in the volume sweep, while a “last image” or an “end image” in the volume sweep may be represented by the second image. In addition, positional information of the probe 14 associated with the start image and the end image may be employed to automatically compute a desirable volume angle, thereby advantageously circumventing disadvantages associated with the presently available techniques.

Referring now to FIG. 5, a front view of a portion of the user interface 34 is illustrated. Reference numeral 112 may be representative of a controls portion of the user interface 34, while reference numeral 36 may be indicative of a volume angle button. The volume angle button 36 may be configured to aid a user, such as the clinician, in selecting a desirable volume angle for the acquisition of image data in a current imaging session. More particularly, the volume angle button 36 may be configured to facilitate the clinician in identifying a desirable number of images. In other words, the volume angle button 36 may be configured to aid the clinician in identifying a desired number of 2D images for forming a desirable three-dimensional (3D) image volume and/or a four-dimensional (4D) image volume. For example, if the clinician selects a volume angle of about 30 degrees, then the imaging system 10 may be configured to facilitate the acquisition of about 30 2D frames, where the acquired 2D images may be employed to generate a 3D image volume. Additionally, as will be appreciated, a range of volume angles may be dependent upon an anatomical region of interest, an application, or both. By way of example, if the anatomical region of interest includes the abdomen, then the volume angle may be in a range from about 18 degrees to about 75 degrees. Similarly, the volume angle for vascular applications may be in a range from about 4 degrees to about 29 degrees, while use of the imaging system for endo-vaginal applications may call for a volume angle in a range from about 6 degrees to about 90 degrees. Furthermore, neo-natal applications may allow a volume angle in a range from about 6 degrees to about 90 degrees.

Further, reference numeral 37 may be representative of a start sweep button, where the start sweep button 37 may be configured to allow the clinician to initiate a volume sweep, thereby triggering the acquisition of image data. In other words, using the start sweep button 37, the clinician may communicate to the imaging system 10 regarding the starting of acquisition of image data. In a similar fashion, a stop sweep button 38 may be configured to aid the clinician in stopping or ending the volume sweep, thereby ending the acquisition of image data. By selecting the stop sweep button 38, the clinician may communicate the cessation of the current acquisition of image data.

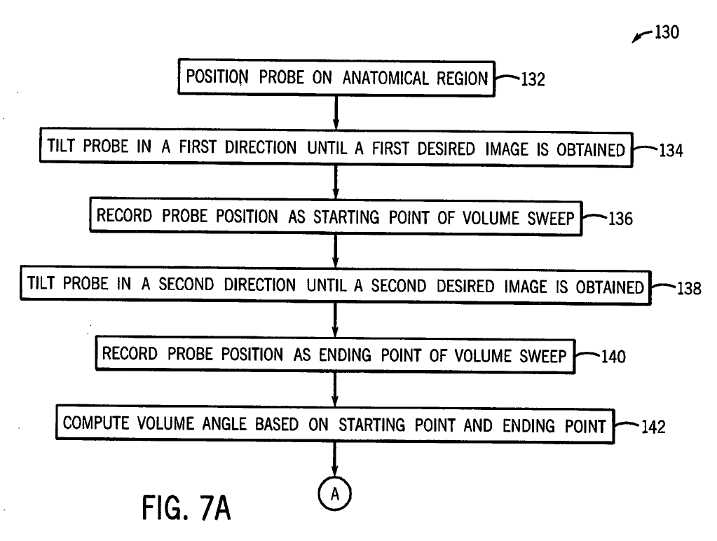

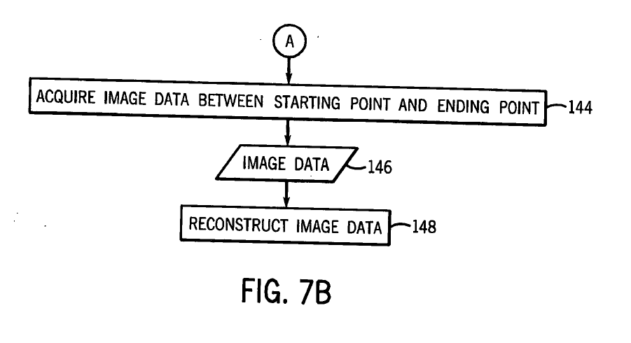

In accordance with further aspects of the present technique, the user interface 34 may also include a select start image button 39 and a select end image button 40. The select start image button 39 may be configured to allow the clinician to select the first desired image, such as a “start image” in the volume sweep. As used herein, the term start image may be used to represent a desired image that is a starting reference point for the volume sweep. In a similar fashion, the select end image button 40 may be configured to allow the clinician to select a second desired image, such as an “end image” in the volume sweep. As used herein, the term end image may be representative of a desired image that is an ending reference point for the volume sweep. The working of the volume angle button 36, the start sweep button 37, the stop sweep button 38, the select start image button 39, and the select end image button 40 will be described in greater detail with reference to FIGS. 7-11.

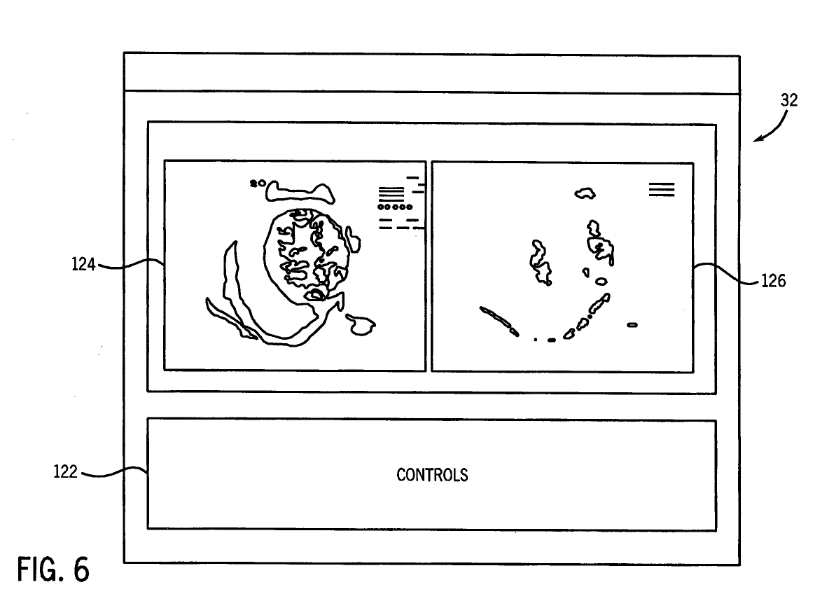

FIG. 6 illustrates a front view of a portion of the display 32 (see FIG. 1). As noted hereinabove, employing the select start image button 39 (see FIG. 5) and the select end image button 40 (see FIG. 5), a first desired image (start image) and a second desired image (last image or end image) may be obtained. Accordingly, the display 32 may be configured to aid in visualizing the first and second desired images. Reference numeral 124 is representative of the first desired image, while the second desired image may be represented by reference numeral 126. Reference numeral 122 may be indicative of a controls portion of the display 32.

As described hereinabove, presently available techniques call for the selection of a volume angle by the clinician prior to the acquisition of image data, where the volume angle may be used to determine the sweep angle of the probe 14. As previously noted, this determination of the sweep angle of the probe 14 may disadvantageously lead to an undesirable volume, thereby resulting in the clinician working with an image volume that is larger than necessary or an image volume that is smaller than a desired image volume. Additionally, a different volume angle may have to be selected and the scan may have to be repeated.

In accordance with exemplary aspects of the present technique, the shortcomings associated with the presently available techniques may be circumvented by acquiring image data based on positional information associated with the probe 14. Accordingly, a method of imaging based on positional information of the probe 14 and a system for imaging based on positional information of the probe 14 are presented.

The working of the diagnostic imaging system 10 (see FIG. 1), and more particularly the position sensing platform 28 (see FIG. 1) may be better understood with reference to the exemplary logic depicted in FIGS. 7-11. Referring now to FIGS. 7A-7B, a flow chart of exemplary logic 130 for imaging an anatomical region of interest based on positional information associated with an image acquisition device, such as the probe 14 (see FIG. 1) is illustrated. In accordance with exemplary aspects of the present technique, a method for imaging based on positional information is presented.

The method starts at step 132 when the clinician positions an image acquisition device, such as the probe 14 (see FIG. 1), on the patient 12 (see FIG. 1). More particularly, the probe 14 may be positioned on an anatomical region of interest. Consequently, the probe 14 is in contact with the patient 12 (see FIG. 1). It may be noted that although the present example is described in terms of 3D imaging, the present technique may also find application in 4D ultrasound imaging. As will be appreciated, a 3D ultrasound imaging system is configured to provide the clinician with a 3D image of an anatomical region of interest in the patient 12. More particularly, the probe in the 3D ultrasound imaging system is configured to obtain a series of 2D images of the patient. Subsequently, the 3D ultrasound imaging system processes these images and presents the images as a 3D image. The clinician may then manipulate the 3D image to obtain views that may not be available using a 2D ultrasound imaging system.

Further, as previously noted with reference to. FIG. 3, the probe 14 may include a curvilinear probe face, where the curvilinear probe face includes the central position 82, the first end 84 and the second end 86. Accordingly, in the p resent example, at step 132, the probe 14 may be positioned on the patient 12 such that the central portion 82 of the probe 14 is in contact with the patient 12. Also, as will be appreciated, while the probe 14 is positioned in the central position an image representative of the anatomical region of interest on the patient 12 may be acquired via the probe 14 and displayed on the display 32 (see FIG. 1) of the imaging system 22 (see FIG. 1). The clinician may then view this image to verify if the image is representative of a desired image.

Subsequently, at step 134, the probe 14 may be moved in a first direction until a first desired image is obtained. Accordingly, the clinician may move the probe 14 in the first direction such that the probe 14 is tilted from the central position 82 (see FIG. 3) towards the first end 84 (see FIG. 3) of the probe 14 until the clinician views the first desired image. As used herein, the term first desired image is used to represent a desirable image representative of the anatomical region of interest. In other words, the first desired image may be representative of a desired “start image”, where the start image may be representative of a starting point in the volume sweep. It may be noted that a position of the probe 14 at the starting point of the volume sweep may be referred to as a “starting position” of the probe 14. Further, at step 134, the clinician may then select that first desired image as a desired start image. In one embodiment, the clinician may record the first desired image as the start image via the select start image button 39 (see FIG. 5), for example.

Once the clinician has selected the first desired image, information associated with a current position of the probe 14 may be recorded, at step 136. For example, the position sensing device 18 (see FIG. 1) may be utilized to record positional coordinates of the probe 14 associated with the first desired image. This position may generally be referred to as a “starting point” of a volume sweep. Also, the start image may be visualized on a portion of the display 32 (see FIG. 6).

Furthermore, at step 138, following the recording of the positional information of the probe 14 corresponding to the first desired image, the clinician may tilt the probe 14 in a second direction, where the second direction is in a direction that is substantially opposite the first direction. In other words, the clinician may tilt the probe 14 such that the probe 14 is moved towards the second end 86 (see FIG. 3) of the probe 14. More particularly, the clinician may tilt the probe 14 in the second direction until a second desired image is obtained. As used herein, the term second desired image is used to represent another desirable image representative of the anatomical region of interest. In other words, the second desired image may be representative of a desired “end image”, where the “end image” may be representative of an ending point in the volume sweep. It may be noted that a position of the probe at the ending point in the volume sweep may generally be referred to as an “ending position” of the probe 14. Additionally, at step 138, the clinician may select that second desired image as a desired end image. The clinician may record the second desired image as the end image via the select end image button 40 (see FIG. 5), in one embodiment. Here again, the end image may be visualized on a portion of the display 32 (see FIG. 6).

Moreover, at step 140, information associated with a current position of the probe 14 may be recorded. For example, the position sensing device 18 (see FIG. 1) may be utilized to record positional coordinates of the probe 14 associated with the second desired image. Also, this position may generally be referred to as an “ending point” of a volume sweep.

Consequent to steps 132-140, the first desired image and the second desired image are obtained, where the first desired image and the second desired image are respectively representative of a “start image” and an “end image” for a current volume sweep. More particularly, subsequent to steps 132-140, the starting point and the ending point of the current volume sweep are obtained. Also, positional information of the probe 14 at each of the positions corresponding to the first desired image and the second desired image is recorded.

Subsequently, at step 142, a sweep angle for the current volume sweep may be computed based on the positional information of the probe 14 recorded at steps 136 and 140. More particularly, information associated with the starting position, the ending position and the central position 82 (see FIG. 3) of the probe 14 in the current volume sweep may be used to automatically compute the sweep angle for the volume sweep.

Once the sweep angle has been determined based on the positional information of the probe 14, image data representative of the anatomical region of interest may be obtained, at step 144. In one embodiment, the clinician may trigger the acquisition of image data. By way of example, the clinician may initiate the acquisition of image data by selecting the start sweep button 37 (see FIG. 5). In accordance with exemplary aspects of the present technique, image data may be obtained between the starting point and the ending point in the current volume sweep recorded at steps 136 and 140 respectively. Accordingly, the probe 14 may be returned to the central position 82. The probe 14 may then be moved in the first direction towards the starting point of the volume sweep determined at step 136.

As previously described with reference to FIG. 4, the position sensor processing module 94 (see FIG. 4) may be configured to obtain the positional information of the probe 14 recorded at step 136 and step 140. Once the acquisition of image data is triggered, the position sensor processing module 94 may be configured to communicate the positional information associated with the starting point and the ending point of the probe 14 to the probe motor controller 102 (see FIG. 4). In one embodiment, the probe motor controller 102 may then be configured to facilitate automatically moving the probe 14 to the starting point of the volume sweep. Alternatively, the clinician may manually move the probe 14 to the starting position.

Additionally, image data may then be acquired as the probe 14 is swept from the starting point towards the ending point of the image volume. As will be appreciated, incremental angles in the volume sweep may be specified by the clinician prior to the acquisition of image data. Alternatively, the imaging system 22 may be configured to provide default settings of the incremental angles based on the anatomical region of interest. Accordingly, the probe 14 may be incrementally swept from the starting point through the ending point thereby acquiring a plurality of intermediate images between the start image and the end image. Once the end image is obtained, the current volume sweep may be stopped. In one embodiment, the clinician may end the current volume sweep by selecting the stop sweep button 38 (see FIG. 5).

Consequent to the acquisition step 144, image data 146 representative of the anatomical region of interest may be obtained. Further, at step 148, following the acquisition of image data 146, the image data 146 may be subject to one or more processing steps to facilitate reconstruction of the image data 146 to generate an image representative of the anatomical region of interest. The reconstructed image may include a 3D image, in certain embodiments. Moreover, the reconstructed image may then be displayed on the display 32, for example. Additionally, the reconstructed image may also be stored for further use.

By implementing the diagnostic imaging system 10 and method of imaging as described hereinabove, a desired image volume may be obtained as the image volume is acquired between the start image and the end image selected by the clinician, thereby enhancing the efficiency and speed of the imaging process. More particularly, the method of imaging allows the clinician to preview the start and end images ahead of the scan, thereby ensuring acquisition of a desirable image volume. Also, it may be noted that no image data is acquired during the preview process. In addition, the sweep angle is automatically determined based on the positional information associated with the probe 14. Moreover, patient discomfort may be substantially reduced as the system has prior knowledge of the desired image volume and hence the patient breath hold time may be substantially reduced.

Furthermore, as previously noted, the presently available techniques typically entail the selection of a volume angle by the clinician, where the selected volume angle is used to compute the sweep angle of the probe 14. This determination may disadvantageously lead to the acquisition of an undesirable image volume, and may necessitate one or more repeat scans, thereby causing discomfort to the patient 12 and/or a laborious, time-consuming process. Also, based on the computed sweep angle, the currently available techniques typically sweep the probe 14 (see FIG. 1) symmetrically about the central position 82 (see FIG. 3) of the probe 14. For example, if the computed sweep angle is about 40 degrees, then the current techniques are configured to sweep the probe 14 symmetrically about the central position 82 of the probe 14. In other words, the probe may be swept 20 degrees towards the first end 84 (see FIG. 3) of the probe 14 and 20 degrees towards the second end 86 (see FIG. 3) of the probe 14. This symmetrical sweep of the probe 14 in presently available techniques may disadvantageously lead to the acquisition of an undesirable image volume and/or omission of a desirable image volume.

However, in accordance with aspects of the present technique, the sweep angle is computed based on positional information associated with the probe 14, thereby advantageously facilitating acquisition of a desired image volume and circumventing the shortcomings of the currently available techniques. In addition, in the present technique, the probe 14 may also be configured to be swept in an asymmetrical fashion about the central position 82 of the probe 14. For example, using the present technique, based on the start and end images selected by the clinician, if the computed sweep angle is about 40 degrees, the probe 14 may be configured to be swept towards the first end 84 of the probe 14 by about 30 degrees, and by about 10 degrees towards the second end 86 of the probe 14. This asymmetric sweep of the probe 14 about the central position 82 of the probe 14 advantageously facilitates acquisition of a desirable image volume that has been selected by the clinician.

It may also be noted that, in accordance with further aspects of the present technique, if the imaging session includes a 4D scan, positional information obtained via the position sensing device 18 (see FIG. 1) may also be used to notify the clinician if the probe 14 (see FIG. 1) is positioned outside an anatomical region of interest currently being investigated.

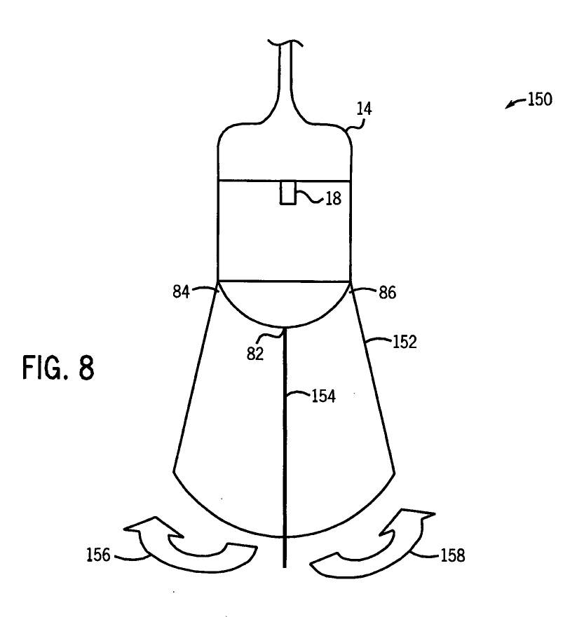

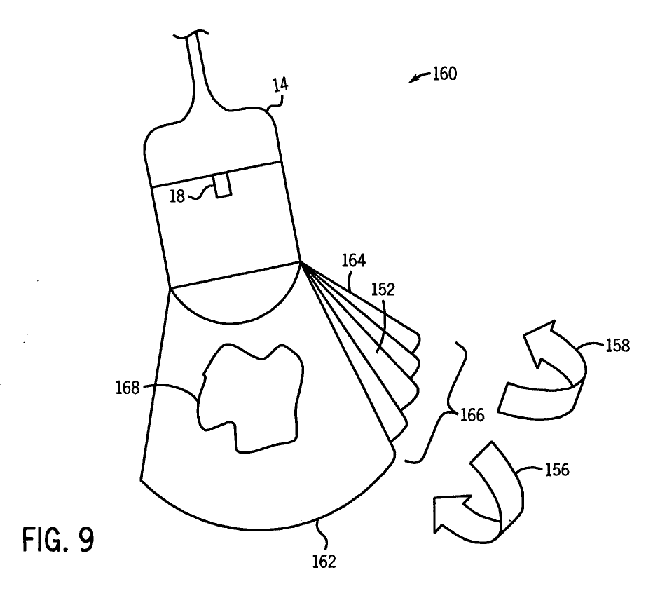

The method of imaging based on positional information of the probe depicted in 132-148 (see FIGS. 7A-7B) may be better understood with reference to FIGS. 8-9. In the example illustrated in FIGS. 8-9, the method of imaging based on positional information of the probe 14 is depicted.

Turning now to FIG. 8, a diagrammatic illustration 150 of the probe 14 (see FIG. 1) is illustrated. An image slice acquired by the probe 14 may be generally represented by reference numeral 152. More particularly, reference numeral 152 may be representative of an image acquired by the probe 14 while positioned in the central position 82 (see FIG. 3) of the probe 14. Also, the reference numeral 154 may generally be representative of a line along the central position 82 of the probe 14. Further, reference numeral 156 may be indicative of a first direction of moving the probe 14. More particularly, the first direction 156 is representative of a direction of moving the probe 14 from the central position 82 towards the first end 84 (see FIG. 3) of the probe 14. In a similar fashion, a second direction of moving the probe 14 may generally be represented by reference numeral 158. More particularly, the second direction 158 is representative of a direction of moving the probe 14 from the central position 82 towards the second end 86 (see FIG. 3) of the probe 14.

Referring now to FIG. 9, a diagrammatic illustration 160 of the method of imaging based on the positional information associated with the probe 14 (see FIG. 1) is illustrated. As previously described with reference to FIGS. 7A-7B, the probe 14 may be positioned on the patient 12 (see FIG. 1) to facilitate imaging an anatomical region of interest. More particularly, the probe 14 may be positioned on the patient 12 such that the central portion 82 (see FIG. 3) of the probe 14 is in contact with the patient 12. Further, the image 152 (see FIG. 8) may be visualized on the display 32 (see FIG. 1). In accordance with exemplary aspects of the present technique, the probe 14 may be moved in the first direction 156 such that the probe 14 is moved from the central position 82 towards the first end 84 (see FIG. 3) of the probe 14. As the probe 14 is moved in the first direction 156, the clinician may visualize corresponding images of the anatomical region of interest on the display 32. Once the clinician identifies an image as the first desired image, the clinician may communicate the selection of an image as the first desired image to the imaging system 22 (see FIG. 1). In the present example, the clinician may choose the first desired image by selecting the select start image button 39 (see FIG. 5). In the example illustrated in FIG. 9, reference numeral 162 may be representative of the first desired image or the start image. Further, the start image may be visualized on a portion of the display 32 (see FIG. 6).

Also, positional information of the probe 14 associated with the start image may be recorded as the first position or starting position of the probe 14 in the volume sweep. Information corresponding to the starting position of the probe 14 may be obtained via use of the position sensing device 18 (see FIG. 1). More particularly, the position sensing transmitter 98 (see FIG. 4) may be employed to communicate the starting position information to the position sensor receiver 42 (see FIG. 4). The position sensor receiver 42 may then communicate the received positional information to the position sensing platform 28 (see FIG. 1), and more particularly to the position sensor processing module 94 (see FIG. 4).

Subsequent to the identification and selection of the start image and the recordation of the starting position of the probe 14, the probe 14 may be moved in the second direction 158. In other words, the probe 14 may be moved in the second direction 158 from the starting point towards the second end 86 of the probe 14. More particularly, the probe 14 may be moved in the second direction 158 until a second desired image is obtained. Here again, as the probe 14 is moved in the second direction 158, the clinician may visualize corresponding images of the anatomical region of interest on the display 32. The clinician may then identify an image as the second desired image. Further, in one embodiment, the clinician may communicate the selection of an image as the second desired image to the imaging system 22 (see FIG. 1) by selecting the select end image button 40 (see FIG. 5). Also, in the present example, reference numeral 164 may be representative of the second desired image or the end image. The end image 164 may be visualized on a portion of the display 32 (see FIG. 1), as previously noted.

Moreover, positional information of the probe 14 associated with the end image may be recorded as the second position or ending position of the probe 14 in the volume sweep. For example, information corresponding to the ending position of the probe 14 may be obtained via use of the position sensing device 18. Here again, the position sensing transmitter 98 (see FIG. 4) may be employed to communicate the ending position in formation to the position sensor receiver 42 (see FIG. 4). The position sensor receiver 42 may then communicate the received positional information to the position sensing platform 28, and more particularly to the position sensor processing module 94.

Subsequent to the acquisition of the start image 162 and the end image 164 and the corresponding starting position and ending position of the probe 14 in the current volume sweep, a sweep angle for the probe 14 may be computed. As previously noted, the sweep angle may be automatically computed based on the information associated with the starting and ending positions of the probe 14 in the current volume sweep. In one embodiment, the position sensor processing module 94 may be configured to aid in the automatic computation of the sweep angle for the probe 14 based on the positional information associated with the starting position and ending position of the probe 14.

The computed sweep angle may then be communicated to the probe 14. More particularly, the position sensor processing module 94 may be configured to facilitate the communication of the computed sweep angle and positional information associated with the starting and ending positions of the probe 14 to the probe motor controller 102 (see FIG. 4), in one embodiment.

Once the acquisition of image data is initiated, by the clinician for example, the probe motor controller 102 may be configured to move the probe 14 to the previously determined starting position. As previously noted, the acquisition of image data may be triggered by selecting the start s weep button 37 (see FIG. 5). Furthermore, as will be appreciated, at the starting position, the first desired image or the start image 162 may be obtained. In addition, the probe motor controller 102 may be configured to automatically sweep the probe 14 between the predetermined starting position and the ending position. In one embodiment, the probe 14 may be swept between the starting and ending positions in incremental steps, where the incremental steps may be selected by the clinician or may be determined by the imaging system 22 based on the anatomical region of interest. Image data representative of the anatomical region of interest at each incremental step may be acquired beginning at the starting position of the probe 14 until the previously determined ending position of the probe 14 is reached. In other words, a plurality of images may be obtained at each incremental step of probe 14 between the previously determined start and end images. Reference numeral 166 may be representative of intermediate images obtained between the start image 162 and the end image 164. Also, the anatomical region of interest may generally be represented by reference numeral 168.

The plurality of images 162, 164, 166 so obtained by sweeping the probe 14 between the starting point and ending point of the probe 14 may then be processed to reconstruct an image representative of the anatomical region of interest. In the present example, the plurality of images 162, 164, 166 may be reconstructed to generate a 3D image representative of the anatomical region of interest.

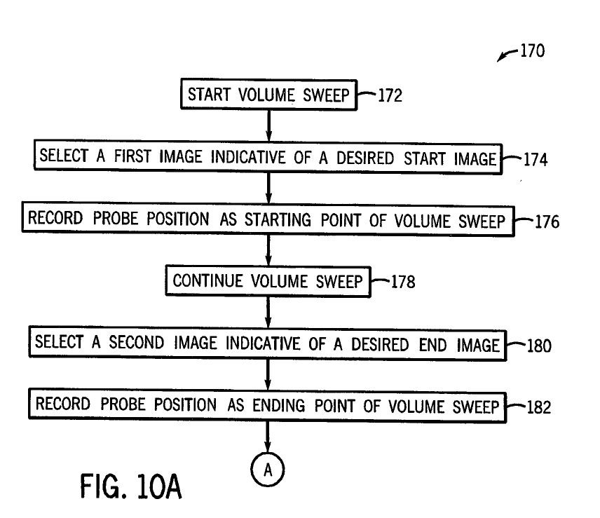

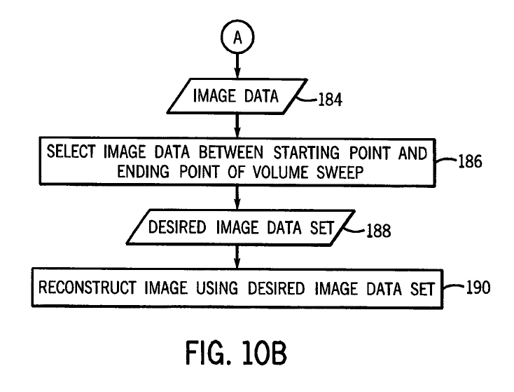

In accordance with exemplary aspects of the present technique, another method for imaging based on positional information is presented. FIGS. 10A-10B are representative of a flow chart of another exemplary logic 170 for imaging an anatomical region of interest based on positional information associated with an image acquisition device, such as the probe 14 (see FIG. 1).

The method starts at step 172 when the clinician positions an image acquisition device, such as the probe 14 (see FIG. 1), on the patient 12 (see FIG. 1). In addition, the clinician may start the sweep of the probe 14. For example, in one embodiment, the sweep of the probe 14 may be started from the first end 84 (see FIG. 3 or FIG. 8) and continued towards the second end 86 (see FIG. 3 or FIG. 8) of the probe 14. Alternatively, in another embodiment, the sweep of the probe 14 may begin at the second end 86 and continued towards the first end 84 of the probe 14.

In accordance with aspects of the present technique, as the sweep is initiated from the first end 84 towards the second end 86, a first desired image indicative of a “start image” may be selected, at step 174. For example, as the probe is s wept starting at the first end 84 of the probe, the clinician while viewing the images on the display 32 (see FIG. 1) may select an image as the start image. Information associated with the probe position during the start image may also be recorded as depicted in step 176. This position of the probe 14 may be indicative of a first position or a “starting position” of the probe 14. Also, the start image may be displayed on a first portion of the display, as depicted in FIG. 6.

Subsequently, as indicated by step 178, the sweep of the probe 14 may be continued after the selection of the start image and the recordation of the starting position of the probe 14. Further, at step 180, a second desired image may then be selected as an “end image” for the volume sweep. Here again, as the probe 14 is swept towards the second end 86 of the probe 14, the clinician may select a second desired image as the end image. In addition, information associated with the probe position during the end image may also be recorded as depicted in step 182. This position of the probe 14 may be indicative of a second position or a “ending position” of the probe 14. Also, the end image may be displayed on a second portion of the display, as depicted in FIG. 6. It may be noted that in the present example, image data representative of the anatomical region of interest is acquired as the probe 14 is swept from the starting point to the ending point in the current volume sweep.

Subsequent to steps 172-182, as the probe 14 is swept between the first end 84 of the probe 14 and the ending point of the volume sweep, image data 184 representative of the anatomical region of interest may be obtained. The image data 184 so acquired may include image data corresponding to probe positions starting at the first end 84 of the probe 14 and ending at the ending point 86 of the probe 14. More particularly, as the probe 14 is incrementally swept from the first end 84 of the probe 14 through the ending point 86, a plurality of images may be obtained.

Subsequently, at step 186, a desired image data set corresponding to image data between the starting position and the ending position of the probe 14 may be selected. In other words, the desired image data set so selected at step 186 may be configured to include image data corresponding to the start image, the end image and intermediate images therebetween. The desired image data set may generally be represented by reference numeral 188. Further, at step 190, the desired image data set 188 may then be subject to one or more processing steps to reconstruct the desired image data set 188 to generate an image representative of the anatomical region of interest. This reconstructed image may then be visualized on the display 32, for example. Additionally, the reconstructed image may also be stored for further use.

In one embodiment, the reconstructed images may be stored in the data repository 30 (see FIG. 1). In certain embodiments, the data repository 30 may include a local database. Alternatively, these images may be stored in an archival site, a database, or an optical data storage article. For example, the reconstructed images may be stored in the optical data storage article. It may be noted that the optical data storage article may be an optical storage medium, such as a compact disc (CD), a digital versatile disc (DVD), multi-layer structures, such as DVD-5 or DVD-9, multi-sided structures, such as DVD-10 or DVD-18, a high definition digital versatile disc (HD-DVD), a Blu-ray disc, a near field optical storage disc, a holographic storage medium, or another like volumetric optical storage medium, such as, for example, two-photon or multi-photon absorption storage format. Furthermore, these reconstructed images may be stored locally on the medical imaging system 22 (see FIG. 1).

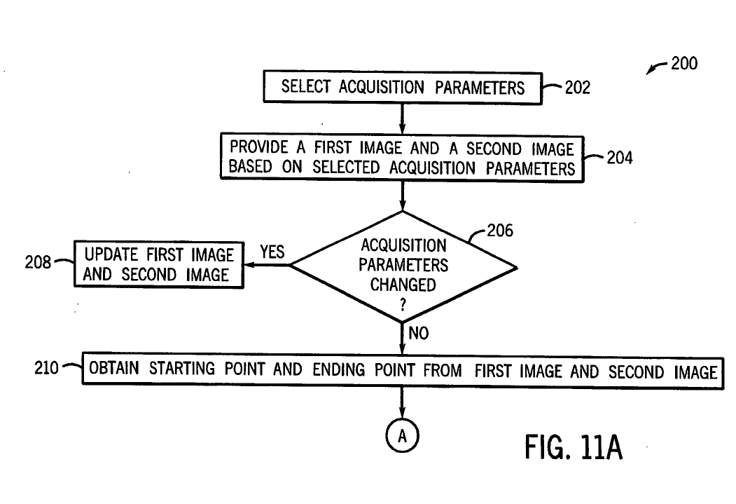

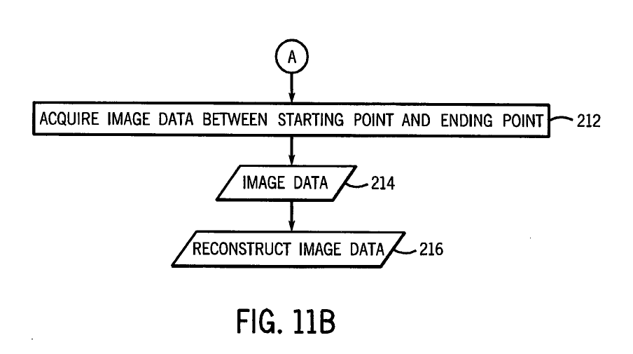

In accordance with exemplary aspects of the present technique, yet another method for imaging based on positional information is presented. Referring now to FIGS. 11A-11B, a flow chart of yet another exemplary logic 200 for imaging an anatomical region of interest based on positional information associated with an image acquisition device, such as the probe 14 (see FIG. 1) is depicted.

The method starts at step 202, where acquisition parameters associated with a current imaging session may be selected. The acquisition parameters may include a volume angle, a sweep angle, quality, depth, or region of interest, for example. Once the acquisition parameters are selected, the imaging system 22 (see FIG. 1) may be configured to provide the clinician with a first image and a second image, where the first image and the second image are obtained based on the selected acquisition parameters, at step 204. Further, the first image so obtained may be representative of a start image of the volume sweep, while the second image may be representative of an “end image” of the volume sweep. For example, following the selection of the volume angle by the clinician, the imaging system 22 may be configured to provide a first image and a second image that are generated by the imaging system 22 based on the selected volume angle. More particularly, if the clinician selects a volume angle of about 30 degrees, the imaging system 22 (see FIG. 1) may be configured to move the probe 14 (see FIG. 1) from the central position 82 (see FIG. 3) of the probe 14 in the first direction 156 (see FIG. 8) towards the first end 84 (see FIG. 3) to a position that is about 15 degrees in the first direction 156. The first image at this position of the probe 14 may be obtained as the start image of the volume sweep. Also, positional coordinates of the probe 14 associated with the start image may be obtained and recorded as a “starting point” of the volume sweep. As previously noted, the starting point of the volume sweep may be indicative of a starting position of the probe 14 in the current volume sweep.

With continuing reference to step 204, subsequently, the probe 14 may be moved in the second direction 158 (see FIG. 8) towards the second end 86 (see FIG. 3) of the probe 14 to a position that is about 15 degrees in the second direction 158. The second image at this position of the probe 14 may be obtained as the “end image” of the volume sweep. Here again, positional coordinates of the probe 14 associated with the end image may be obtained and recorded as an “ending point” of the volume sweep. The ending point of the volume sweep may be representative of an ending position of the probe 14, as previously described. The imaging system 22 may then be configured to display the start image and the end image on the display 32 (see FIG. 6).

In accordance with aspects of the present technique, if desired start and end images are not visualized, the clinician may change the volume angle. In other words, if the start and end images are not representative of desirable images, the clinician may appropriately change the volume angle. First and second images based on the updated volume angle may then be obtained to serve as the updated start and end images respectively. Also, in accordance with further aspects of the present technique, the first and second images may also be updated based on any revisions of other acquisition parameters. Accordingly, a check may be carried out at step 206 to verify if one or more acquisition parameters have been changed. If a change in one or more acquisition parameters is detected, then updated first and second images may be obtained based on the updated acquisition parameters, as indicated by step 208.

Subsequently, information associated with the starting position and the ending position of the probe 14 may be obtained at step 210. Also, at step 212, image data may be acquired between the starting point and the ending point recorded at step 210. In other words, the probe 14 may be moved to the starting position of the probe 14 and image data may be acquired as the probe 14 is swept from the starting position to the ending position of the probe 14. Reference numeral 214 may be representative of the acquired image data. This acquired image data 214 may then be reconstructed to generate an image volume representative of the anatomical region of interest, at step 216. However, at decision block 206, if no change in the acquisition parameters is detected, steps 210-216 may be carried out.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, including but not limited to C++ or Java. Such code, as will be appreciated by those of ordinary skill in the art, may be stored or adapted for storage on one or more tangible, machine readable media, such as on memory chips, local or remote hard disks, optical disks (that is, CDs or DVDs), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

The method of imaging based on positional information associated with the image acquisition device and the system for imaging described hereinabove simplify procedural workflow for imaging an anatomical region of interest in the patient and enhance the speed of procedural time taken to image the anatomical region of interest in the patient. Further, the method allows the clinician to preview the start image and the end image in the volume sweep, thereby facilitating acquisition of only a desirable volume of interest. Furthermore, the method involves previewing the start and end images of the image volume, without acquiring the volume. Consequently, only the desired amount of image data may be collected, thereby reducing the amount of image data acquired and enhancing system response. Furthermore, as the method of imaging entails acquisition of only a desirable volume of interest, patient breath hold time is reduced, thereby enhancing patient comfort and reducing breathing artifacts. In addition, for treatment monitoring studies, position sensor data may be used to reproduce substantially similar volume images.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for imaging based on a position of an image acquisition device, the method comprising:
obtaining a first desired image data set representative of a first desired image, wherein the first desired image data set is acquired at a first position of the image acquisition device;
recording positional information corresponding to the first position of the image acquisition device;
obtaining a second desired image data set representative of a second desired image, wherein the second desired image data set is acquired at a second position of the image acquisition device;
recording positional information corresponding to the second position of the image acquisition device; and
acquiring image data between the first position and the second position of the image acquisition device.

2. The method of claim 1, wherein obtaining the first desired image data set comprises moving the image acquisition device in a first direction to facilitate acquisition of the first desired image data set.

3. The method of claim 2, wherein obtaining the second desired image set comprises moving the image acquisition device in a second direction to facilitate acquisition of the second desired image data set, wherein the second direction is opposite the first direction.

4. The method of claim 1, wherein the image acquisition device comprises a probe, wherein the probe comprises an imaging catheter, an endoscope, a laparoscope, a surgical probe, an external probe, or a probe adapted for interventional procedures.

5. The method of claim 1, wherein the first position of the image acquisition device is a starting point of a volume sweep of the image acquisition device, and the second position of the image acquisition device is an ending point of the volume sweep of the image acquisition device.

6. The method of claim 1, further comprising positioning the image acquisition device on an anatomical region of interest on a patient.

7. The method of claim 1, further comprising displaying the first desired image and the second desired image on a display.

8. The method of claim 1, wherein acquiring image data between the first position and the second position of the image acquisition device comprises:
steering the image acquisition device to the first position;
acquiring image data starting at the first position of the image acquisition device; and
continuing acquisition of image data until the second position of the image acquisition device.

9. The method of claim 8, further comprising reconstructing the acquired image data to generate a user-viewable representation of the acquired image data.

10. The method of claim 1, further comprising selecting acquisition parameters.

11. The method of claim 10, further comprising sensing changes in the acquisition parameters.

12. The method of claim 11, further comprising generating an updated first desired image and an updated second desired image based on the changed acquisition parameters.

13. A method for imaging based on a position of an image acquisition device, the method comprising:
selecting acquisition parameters;
obtaining a first desired image and a second desired image based on the selected acquisition parameters; and
recording a first position and a second position of the image acquisition device, wherein the first position of the image acquisition device is associated with the first desired image and the second position of the image acquisition device is associated with the second desired image; and
acquiring image data between the first position and the second position of the image acquisition device.

14. The method of claim 13, further comprising sensing changes in the acquisition parameters.

15. The method of claim 14, further comprising generating an updated first desired image and an updated second desired image based on the changed acquisition parameters.

16. A computer readable medium comprising one or more tangible media, wherein the one or more tangible media comprise:
code adapted to obtain a first desired image data set representative of a first desired image, wherein the first desired image data set is acquired at a first position of an image acquisition device;
code adapted to record positional information corresponding to the first position of the image acquisition device;
code adapted to obtain a second desired image data set representative of a second desired image, wherein the second desired image data set is acquired at a second position of the image acquisition device;
code adapted to record positional information corresponding to the second position of the image acquisition device; and
code adapted to acquire image data between the first position and the second position of the image acquisition device.

17. The computer readable medium, as recited in claim 16, wherein the code adapted to acquire image data between the first position and the second position of the image acquisition device comprises:
code adapted to steer the image acquisition device to the first position;
code adapted to acquire image data starting at the first position of the image acquisition device; and
code adapted to continue acquisition of image data until the second position of the image acquisition device.

18. The computer readable medium, as recited in claim 16, further comprising code adapted to reconstruct the acquired image data to generate a user-viewable representation of the acquired image data.

19. The computer readable medium, as recited in claim 16, further comprising code adapted to select acquisition parameters.

20. A position sensing system, comprising:
a position sensing platform configured to facilitate acquisition of image data based on a first position and a second position of an image acquisition device, wherein the position sensing platform is configured to:
obtain a first desired image data set representative of a first desired image, wherein the first desired image data set is acquired at the first position of the image acquisition device;
record positional information corresponding to the first position of the image acquisition device;
obtain a second desired image data set representative of a second desired image, wherein the second desired image data set is acquired at the second position of the image acquisition device;
record positional information corresponding to the second position of the image acquisition device; and
acquire image data between the first position and the second position of the image acquisition device.

21. The system of claim 20, further configured to generate a user-viewable representation of the acquired image data.

22. A system for acquiring image data based on a position of an image acquisition device, the system comprising:
an image acquisition device configured to acquire image data representative of an anatomical region of interest;
a position sensing device in operative association with the image acquisition device and configured to provide positional information associated with the image acquisition device;
an imaging system in operative association with the image acquisition device and comprising:
an acquisition subsystem configured to acquire image data, wherein the image data is representative of the anatomical region of interest; and
a processing subsystem in operative association with the acquisition subsystem and comprising a position sensing platform configured to facilitate moving the image acquisition device to at least a first desirable position and a second desirable position based on the acquired image data and positions of the image acquisition device.

23. The system of claim 22, wherein the imaging system comprises an ultrasound imaging system.

24. The system of claim 22, wherein the position sensing platform is further configured to:
obtain a first desired image data set representative of a first desired image, wherein the first desired image data set is acquired at the first desirable position of an image acquisition device;
record positional information corresponding to the first desirable position of the image acquisition device;
obtain a second desired image data set representative of a second desired image, wherein the second desired image data set is acquired at the second desirable position of the image acquisition device;
record positional information corresponding to the second desirable position of the image acquisition device; and
acquire image data between the first desirable position and the second desirable position of the image acquisition device.

25. The system of claim 22, wherein the position sensing device comprises a position sensor configured to provide location information of the image acquisition device.
